(19) United States
(12) Patent Application Publication
(10) Pub. No.: US 2002/0168645 A1
(43) Pub. Date: Nov. 14, 2002

(54) Analysis of polynucleotide sequence

(75) Inventor: Seth Taylor, Cambridge, MA (US)

Correspondence Address: LOUIS MYERS, Fish & Richardson P.C., 225 Franklin Street, Boston, MA, 02110-2804, US

(73) Assignee: Seth Taylor

(21) Appl. No.: 09/884,425

(22) Filed: Jun. 19, 2001

Related U.S. Application Data
(63) Continuation of application No. 09/293,333, filed on Apr. 16, 1999, now abandoned.
(60) Provisional application No. 60/082,063, filed on Apr. 16, 1998.
(60) Provisional application No. 60/084,085, filed on May 4, 1998.

(51) Int. Cl. C12Q 1/68

(52) U.S. Cl. 435/6

(57) ABSTRACT

Disclosed are methods for detecting nucleic acids using rolling circle-based amplification and arrays of capture probes.

### CROSS REFERENCE TO RELATED APPLICATIONS

This application claims priority from U.S. Ser. No. 60/082,063, filed Apr. 16, 1998, and U.S. Ser. No. 60/084,085, filed May 7, 1998. The contents of these applications are incorporated herein in their entirety.

### BACKGROUND OF THE INVENTION

The invention relates to isothermal methods or analyzing a polynucleotide sequence.

### SUMMARY OF THE INVENTION

In general, the invention includes methods which combine isothermal methods of nucleic acid amplification with a positional array analysis. In some embodiments, the array is a three dimensional array, e.g., a gel pad array, analysis. In preferred methods, a target is isothermally amplified, and the amplification product is contacted with a positional array, thereby analyzing a nucleic acid sequence. Examples of isothermal amplification include, rolling circle amplification, nucleic acid sequence-based amplification (NASBA) (see, e.g., U.S. Pat. Nos. 5,409,818 and 5,130,238), self sustained sequence replication (3SR), strand displacement amplification (SDA) (see, e.g., U.S. Pat. Nos. 5,523,204; 5,455,166; 5,631,147; 5,712,124, and 5,733,752), cycling probe reaction or TMA, (see, e.g., U.S. Pat. Nos. 5,554,516; 5,480,784; and 5,399,491).

The method can also be used to classify a sample in which the nucleic acid is or was found.

In one aspect, the invention includes a method of analyzing a polynucleotide, e.g., detecting a genetic event, e.g., a single nucleotide polymorphism, in a sample. The method includes:

providing a sample which includes a sample polynucleotide sequence to be analyzed;

(2) (a) annealing an effective amount of sample sequence to a single-stranded circular template to yield an annealed circular template, wherein the single-stranded circular template comprises (i) at least one copy of a nucleotide sequence complementary to the sequence of the sample sequence and optionally, (ii) at least one nucleotide effective to produce a cleavage site in an oligonucleotide multimer;

(b) providing the primed circular template with effective amounts of a primer, at least two types of nucleotide triphosphates and a polymerase enzyme, to yield a single-stranded oligonucleotide multimer complementary to the circular oligonucleotide template, wherein the oligonucleotide multimer comprises multiple copies (amplified) of the sample sequence; optionally,

(c) cleaving the oligonucleotide multimer at the cleavage site to produce the cleaved amplified sample nucleic acid; and

(3) analyzing the sample sequence from (2) (b) or (c), e.g., by providing an array of a plurality of capture probes, wherein each of the capture probes is positionally distinguishable from other capture probes of the plurality on the array, and wherein each positionally distinguishable capture probe of the plurality includes a unique (i.e., not repeated in another capture probe) region; and hybridizing the amplified sample sequence with the array of capture probes, thereby analyzing the sample sequence.

In preferred embodiments, the amplified sequence from step 2 of the method can be further amplified, e.g., amplified by rolling circle, e.g., prior to analysis under step 3. In such embodiments, the amplified sample nucleic acid from step 2, e.g., a cleaved amplified sample nucleic acid, can be amplified further. The second or other subsequent rolling circle amplification can use a circular oligonucleotide probe of the same or similar sequence that used as Step 2, or one of a different sequence. It is also possible that the circular oligonucleotide in a second or subsequent rolling circle amplification, can be, for example, closed or open circular template.

In preferred embodiments, the circular oligonucleic template (of any step) is prepared by a process comprising the steps of:

(a) hybridizing each end of a linear precursor oligonucleotide to a single positioning oligonucleotide, e.g., a sample sequence, having a 5′ nucleotide sequence complementary to a portion of the sequence comprising the 3′ end of the linear precursor oligonucleotide and a 3′ nucleotide sequence complementary to a portion of the sequence comprising the 5′ end of the linear precursor oligonucleotide, to yield an open oligonucleotide circle wherein the 5′ end and the 3′ end of the open circle are positioned so as to abut each other; and

(b) joining the 5′ end and the 3′ end of the open oligonucleotide circle to yield a circular oligonucleotide template. Rolling circle amplification can be primed by the positioning oligonucleotide, e.g., the target nucleic acid, or by another primer, in this or other methods disclosed herein.

In preferred embodiments, analyzing a nucleic acid includes, e.g., sequencing the nucleic acid, e.g., by sequencing by hybridization or positional sequencing by hybridization, detecting the presence of, or identifying, a genetic event, e.g., a SNP, in a target nucleic acid, e.g., a DNA.

In preferred embodiments, the genetic event is within 1, 2, 3, 4 or 5 base pairs from the end of the target molecule, or is sufficiently close to the end of the target molecule that a mismatch would inhibit DNA polymerase-based extension from a target/primed circle. In preferred embodiments the inhibition is at least 50, 75, 90 or 99%.

In preferred embodiments, the target is amplified, e.g., by a isothermal or nonisothermal method, e.g., by PCR, prior to contact with a circular template.

In preferred embodiments the circular template includes a site for a type IIS restriction enzyme and the site is positioned, e.g., such that a type IIS restriction binding at the site cleaves adjacent the region which binds the sample sequence or cleaves in the region which binds the sample sequence.

In a preferred embodiment a region of the circular template is complementary to a genetic event, e.g., a mutation or SNP, and hybridizes effectually to sample nucleic acid having the event and sample nucleic acid not having the event.

In preferred embodiments, each of the capture probes has a binding region for a non-specific endonuclease binding site, e.g., a type IIS restriction enzyme binding site, and the method includes:

hybridizing the single stranded target nucleic acid with the capture probe array, (preferably the region of an amplification product which corresponds to the genet c event hybridizes with the variable region of a capture probe);

(optionally) ligating the single stranded target nucleic acid to a strand of the capture probe;

cleaving the single stranded target nucleic acid/capture probe duplex with a non-specific endonuclease, to form a cleaved single stranded target nucleic acid/capture probe duplex, such that a base corresponding to the genetic event is in the single stranded region formed by the cleavage;

extending along the single strand which contains the genetic event with one and preferably with 2, 3, or all 4 labeled chain terminating nucleotides, wherein if more than one labeled chain terminating nucleotide is used each of the chain terminators, e.g., A or C, are distinguishable, such that the incorporation of a chain terminator indicates the presence of a genetic event,

thereby detecting or identifying a genetic event in a target nucleic acid.

In preferred embodiments the polynucleotide sequence is: a DNA molecule: all or part of a known gene; wild type DNA; mutant DNA; a genomic Fragment, particularly a human genomic fragment; a cDNA, particularly a human cDNA.

In preferred embodiments the polynucleotide sequence is: an RNA molecule: nucleic acids derived from RNA transcripts; wild type RNA; mutant RNA, particularly a human RNA.

In preferred embodiments the polynucleotide sequence is: a human sequence; a non-human sequence, e.g., a mouse, rat, pig, primate.

In preferred embodiments the method is performed: on a sample from a human subject; and a sample from a prenatal subject; as part of genetic counseling; to determine if the individual from which the target nucleic acid is taken should receive a drug or other treatment; to diagnose an individual for a disorder or for predisposition to a disorder; to stage a disease or disorder.

In preferred embodiments the capture probes are single stranded probes in an array.

In preferred embodiments the capture probes have a structure comprising a double stranded portion and a single stranded portion in an array.

In preferred embodiments hybridization to the array is detected by mass spectrophotometry, e.g., by MALDI-TOF mass spectrophotometry.

In preferred embodiments probes are selected for minimal crosshybridization with other probes.

In preferred embodiments the amplified sample sequence has attached thereto a first member of a proximity detector pair and hybridization to the array allows the first member to be brought into proximity with a second member to provide a signal.

In a preferred embodiment the amplified sample sequence which hybridizes to a capture probe, or the capture probe, is the substrate of or template for an enzyme mediated reactions. For example, after hybridization to the capture probe, the amplified sample sequence is ligated to the capture probe, or after hybridization it is extended along the capture probe.

In preferred embodiments the method includes one or more enzyme mediated reactions in which a nucleic acid used in the method, e.g., an amplified sample sequence, a capture probe, a sequence to be analyzed, or a molecule which hybridizes thereto, is the substrate or template for the enzyme mediated reaction. The enzyme mediated reaction can be: an extension reaction, e.g., a reaction catalyzed by a polymerase; a linking reaction, e.g., a ligation, e.g., a reaction catalyzed by a ligase; or a nucleic acid cleavage reaction, e.g., a cleavage catalyzed by a restriction enzyme, e.g., a Type IIS enzyme. The amplified sample sequence which hybridizes with the capture probe can be the substrate in an enzyme mediated reaction, e.g., it can be ligated to a strand of the capture probe or it can be extended along a strand of the capture probe. Alternatively, the capture probe can be extended along the hybridized amplified sample sequence. (Any of the extension reactors discussed herein can be performed with labeled, or chain terminating, subunits.) The capture probe duplex can be the substrate for a cleavage reaction. These reactions can be used to increase specificity of the method or to otherwise aid in detection, e.g., by providing a signal.

Methods such as those described in U.S. Pat. Nos. 5,503,980 or 5,631,134, both of which are hereby incorporated by reference, can be used in methods of the invention. In particular, the array and array-related steps recited herein can use methods taught in these patents.

In preferred embodiments, the method includes: providing an array having a plurality of capture probes, wherein each of the capture probes is a) positionally distinguishable from the other capture probes of the plurality and has a unique variable region (not repeated in another capture probe of the plurality), b) has a variable region capable of hybridizing adjacent to the genetic event; and c) has a 3′ end capable of serving as a priming site for extension hybridizing the amplified sample sequence having a genetic event to a capture probe of the array, (preferably the region of the amplified sample sequence having a genetic event hybridizes adjacent to the variable region of a capture probe); and using the 3′ end of the capture probe to extend across the region of genomic nucleic acid having a genetic event with one or more terminating base species, where if more than one is used each species has a unique distinguishable label e.g. label 1 for base A, label 2 for base T, label 3 for base G, and label 4 for base C; thereby analyzing the amplified sample sequence.

In another aspect, the invention includes a method of analyzing a polynucleotide sequence. The method includes:

providing an array e.g., a three-dimensional array, e.g., a gel array, e.g., an array as described herein, of a plurality of single-stranded circular templates, wherein each of the single-stranded circular templates is positionally distinguishable from other single-stranded circular templates of the plurality on the array, and wherein each positionally distinguishable single-stranded circular templates includes a unique (i.e., not repeated in another circular templates) region complementary to sample target;

(a) contacting a sample with the array to effect annealing an effective amount of sample sequence to a single-stranded circular template in said array to yield a primed circular template, wherein the single-stranded circular template comprises (i) at least one copy of a nucleotide sequence complementary to the sequence of the sample sequence and optionally, (ii) at least one nucleotide effective to produce a cleavage site in the oligonucleotide multimer;

(b) combining the primed circular template with an effective amount of at least two types of nucleotide triphosphates and an effective amount of a polymerase enzyme to yield a single-stranded oligonucleotide multimer complementary to the circular oligonucleotide template, wherein the oligonucleotide multimer comprises multiple copies (amplified) of the sample sequence; and, optionally,

(c) cleaving the oligonucleotide multimer at the cleavage site to product the cleaved amplified sample nucleic acid; and analyzing the sample sequence from b or c. In preferred embodiments it is analyzed by providing an array of a plurality of capture probes, wherein each of the capture probes is positionally distinguishable from other capture probes of the plurality on the array, and wherein each positionally distinguishable capture probe includes a unique (i.e., not repeated in another capture probe) region complementary to the plurality of capture probes; and

(d) hybridizing the amplified sample sequence with the array of capture probes, thereby analyzing the sample sequence.

In preferred embodiments, the circular oligonucleic template is prepared by a process comprising the steps of:

(a) hybridizing each end of a linear precursor oligonucleotide to a single positioning oligonucleotide, e.g., a sample sequence, having a 5′ nucleotide sequence complementary to a portion of the sequence comprising the 3′ end of the linear precursor oligonucleotide and a 3′ nucleotide sequence complementary to a portion of the sequence comprising the 5′ end of the linear precursor oligonucleotide, to yield an open oligonucleotide circle wherein the 5′ end and the 3′ end of the open circle are positioned so as to abut each other; and

(b) joining the 5′ end and the 3′ end of the open oligonucleotide circle to yield a circular oligonucleotide template.

In preferred embodiments, the target is amplified, e.g., by PCR, prior to contact with a circular template.

In another aspect, the invention includes a screening and amplification method to identify circular nucleotide sequences that bind to and/or alter the function of proteins or other targets. Circular nucleotide sequences, or open circles, having random sequence and a common known oligonucleotide linker are screened for target binding to generate a population of selected sequences. The linker an act as a primer binding site for further amplification of or as a cleavage site in the multimer copy.

For example, a population of circular nucleotide sequences is generated. The individual circular nucleotide sequences in the population of circular nucleotide sequences can include a randomized domain of DNA or RNA sequence and a known constant domain of DNA or RNA. The known constant or nonrandom domain provides for a binding site for an oligonucleotide primer and a cleavage site for cleaving multimers into oligomers. The randomized domain can contain about 5-1400 bases but more preferably about 5-190 bases. The known constant domain can contain about 5-100 bases but more preferably about 8-40 bases in length. The initial population of circular sequences which is applied to the sample is a mixture of circular sequences having different randomized sequences and having the same known constant domain sequence. The mixture can contain about 1000-10¹³ different circular DNA or RNA sequences and more preferably about 10,000-10¹¹ different circular DNA or RNA sequences. The initial population of circular sequences can be selected for the capacity to affect the structure or function of a target molecule or to bind the target.

The target molecules of the invention can be biomolecules, e.g., proteins, an nucleic acids, e.g., DNA or RNA sequences. The circular sequences are selected for the capacity to bind and/or functionally modify the activity of the biomolecule.

The selected population of circular sequences is amplified by rolling circle application. The amplified population of sequences from the said rolling circle amplification, e.g., can be amplified further. For example, it can be amplified by rolling circle amplification. The second or subsequent amplifications can be done prior to further analysis. The subsequent rolling circle amplifications can use the same or similar circular sequence as was used in the initial R.C.A. or a different circular sequence. It is also possible that the circular sequence can be, for example, from a closed or open circular template.

Amplified circles, or cleavage products thereof are applied to an array of a plurality of capture probes, wherein each of the capture probes is positionally distinguishable from other capture probes of the plurality on the array, and wherein each positionally distinguishable capture probe includes a unique (i.e., not repeated in another capture probe) region complementary to the plurality of selector probes;

hybridizing the amplified sample sequence with the array of capture probes, thereby identifying circular nucleotide sequences that bind to and/or alter the function of proteins or other targets.

The circular vectors can be closed circular vectors, open circular vectors which when brought into contact with the analyte, have abutting ends which can be covalently linked, e.g., ligated.

The invention also provides a composition comprising circular DNA or RNA sequences, or analogs thereof, having a randomized and a nonrandomized domain on a positionally distinguishable array.

Preferably, a circular template has about 15-1500 nucleotides, and more preferably about 24-500 nucleotides and most preferably about 30-150 nucleotides.

The oligonucleotide circular template itself may be constructed of DNA or RNA or analogs thereof. Preferably, the circular template is constructed of DNA. A liquid, e.g., a sample nucleic acid or protein binds to a portion of the circular template and is preferably single-stranded having about 4-50 nucleotides, and more preferably about 6-12 nucleotides.

The polymerase enzyme can be any that effects the synthesis of the multimer, e.g., any polymerase described in U.S. Pat. No. 5,714,320. Generally, the definitions provided for circular vectors and their amplification in U.S. Pat. No. 5,714,320 apply to terms used herein, unless there is a conflict between the terms in which case the meaning provided herein controls. U.S. Pat. No. 5,714,320, and all other U.S. patents mentioned herein are incorporated by reference.

In another aspect, the invention includes a method for analyzing a nucleic acid, e.g. for detecting a SNP in a oligonucleotide, for example, a piece of genomic DNA. The method includes:

a) providing a first oligonucleotide. The first oligonucleotide is linear, single-stranded and includes:

i) optionally, an universal rolling circle amplification primer sequence;

ii) optionally, a polymorphism specific to the sequence;

iii) a region which is complimentary to a portion of the oligonucleotide, which preferably is twelve to twenty nucleotides, directly adjacent to, but not including the SNP; and

iv) a second region which is complimentary to a second portion of the oligonucleotide which contains the SNP and one or more, but preferably four or five complimentary nucleotides;

b) providing a second oligonucleotide,

c) contacting the first oligonucleotide and second oligonucleotide;

d) connecting, e.g. ligating, the ends of the first oligonucleotide together;

e) providing a polymerase, a primer, which may or may not be the target nucleic acid, and the nucleotides necessary for rolling circle amplification to take place.

f) allowing rolling circle amplification to take place on the ligated first oligonucleotide;

g) optionally, cleaving the products of rolling circle amplification; and

h) analyzing the resulting oligonucleotides, thereby analyzing a nucleic acid.

In a preferred embodiment, the oligonucleotide I is, for example, a piece of genomic DNA.

In a preferred embodiment, the oligonucleotide I is, for example, a piece of PCR amplified nucleic acid.

In a preferred embodiment, the linear single stranded oligonucleotide contains a structural element that cleaves the rolling circle amplification product, for example, a self complimentary hair pin.

In a preferred embodiment, an additional short nucleotide is provided which is complimentary to several nucleotides directly adjacent to the SNP, and has a nucleotide directly adjacent to the SNP but not complimentary to it.

In a preferred embodiment, the products of the rolling circle amplification are analyzed, for example by a gel pad.

In preferred embodiments, analyzing a nucleic acid includes, e.g., sequencing the nucleic acid, e.g., by sequencing by hybridization or positional sequencing by hybridization, detecting the presence of, or identifying, a genetic event, e.g., a SNP, in a target nucleic acid, e.g., a DNA.

In preferred embodiments, the genetic event is within 1, 2, 3, 4 or 5 base pairs from the end of the target molecule, or is sufficiently close to the end of the target molecule that a mismatch would inhibit DNA polymerase-based extension from a target/primed circle. In preferred embodiments the inhibition is at least 50, 75, 90 or 99%.

In preferred embodiments, the target nucleic acid is amplified, e.g., by a isothermal or nonisothermal method, e.g., by PCR, prior to contact with a circular template.

In preferred embodiments the circularized first oligonucleotide provides a circular template which includes a site for a type IIS restriction enzyme and the site is positioned, e.g., such that a type IIS restriction binding at the site cleaves adjacent the region which binds the sample sequence or cleaves in the region which binds the sample sequence.

In a preferred embodiment a region of the circular template is complementary to a genetic event, e.g., a mutation or SNP, and hybridizes effectually to sample nucleic acid having the event and sample nucleic acid not having the event.

In preferred embodiments, the oligonucleotides are amplified by rolling circle amplification, after which the amplified product is annealed to an array of capture probes. Each of the capture probes has a binding region for a non-specific endonuclease binding site, e.g., a type IIS restriction enzyme binding site. The method includes:

hybridizing the single stranded target nucleic acid with the capture probe array, (preferably the region of an amplification product which corresponds to the genetic event hybridizes with the variable region of a capture probe);

(optionally) ligating the single stranded target nucleic acid to a strand of the capture probe;

cleaving the single stranded target nucleic acid/capture probe duplex with a non-specific endonuclease, to form a cleaved single stranded target nucleic acid/capture probe duplex, such that a base corresponding to the genetic event is in the single stranded region formed by the cleavage;

extending along the single strand which contains the genetic event with one and preferably with 2, 3, or all 4 labeled chain terminating nucleotides, wherein if more than one labeled chain terminating nucleotide is used each of the chain terminators, e.g., A or C, are distinguishable, such that the incorporation of a chain terminator indicates the presence of a genetic event.

thereby detecting or identifying a genetic event in a target nucleic acid.

In preferred embodiments the polynucleotide sequence is: a DNA molecule: all or part of a known gene; wild type DNA; mutant DNA; a genomic fragment, particularly a human genomic fragment; a cDNA, particularly a human cDNA.

In preferred embodiments the polynucleotide sequence is: an RNA molecule: nucleic acids derived from RNA transcripts; wild type RNA; mutant RNA, particularly a human RNA.

In preferred embodiments the polynucleotide sequence is: a human sequence; a non-human sequence, e.g., a mouse, rat, pig, primate.

In preferred embodiments the method is performed: on a sample from a human subject; and a sample from a prenatal subject; as part of genetic counseling; to determine if the individual from which the target nucleic acid is taken should receive a drug or other treatment; to diagnose an individual for a disorder or for predisposition to a disorder; to stage a disease or disorder.

In preferred embodiments the capture probes are single stranded probes in an array.

In preferred embodiments the capture probes have a structure comprising a double stranded portion and a single stranded portion in an array.

In preferred embodiments hybridization to the array is detected by mass spectrophotometry, e.g., by MALDI-TOF mass spectrophotometry.

In preferred embodiments probes are selected for minimal crosshybridization with other probes.

In preferred embodiments the amplified sample sequence has attached thereto a first member of a proximity detector pair and hybridization to the array allows the first member to be brought into proximity with a second member to provide a signal.

In a preferred embodiment the amplified sample sequence which hybridizes to a capture probe, or the capture probe, is the substrate of or template for an enzyme mediated reactions. For example, after hybridization to the capture probe, the amplified sample sequence is ligated to the capture probe, or after hybridization it is extended along the capture probe.

In preferred embodiments the method includes one or more enzyme mediated reactions in which a nucleic acid used in the method, e.g., an amplified sample sequence, a capture probe, a sequence to be analyzed, or a molecule which hybridizes thereto, is the substrate or template for the enzyme mediated reaction. The enzyme mediated reaction can be: an extension reaction, e.g., a reaction catalyzed by a polymerase; a linking reaction, e.g., a ligation, e.g., a reaction catalyzed by a ligase; or a nucleic acid cleavage reaction, e.g., a cleavage catalyzed by a restriction enzyme, e.g., a Type IIS enzyme. The amplified sample sequence which hybridizes with the capture probe can be the substrate in an enzyme mediated reaction, e.g., it can be ligated to a strand of the capture probe or it can be extended along a strand of the capture probe. Alternatively, the capture probe can be extended along the hybridized amplified sample sequence. (Any of the extension reactors discussed herein can be performed with labeled, or chain terminating, subunits.) The capture probe duplex can be the substrate for a cleavage reaction. These reactions can be used to increase specificity of the method or to otherwise aid in detection, e.g., by providing a signal.

Methods such as those described in U.S. Pat. Nos.5,503,980 or 5,631,134, both of which are hereby incorporated by reference, can be used in methods of the invention. In particular, the array and array-related steps recited herein can use methods taught in these patents.

In preferred embodiments, the method includes: providing an array having a plurality of capture probes, wherein each of the capture probes is a) positionally distinguishable from the other capture probes of the plurality and has a unique variable region (not repeated in another capture probe of the plurality), b) has a variable region capable of hybridizing adjacent to the genetic event; and c) has a 3′ end capable of serving as a priming site for extension hybridizing the amplified sample sequence having a genetic event to a capture probe of the array, (preferably the region of :he amplified sample sequence having a genetic event hybridizes adjacent to the variable region of a capture probe); and using the 3′ end of the capture probe to extend across the region of genomic nucleic acid having a genetic event with one or more terminating base species, where if more than one is used each species has a unique distinguishable label e.g. label 1 for base A, label 2 for base T, label 3 for base G, and label 4 for base C; thereby analyzing the amplified sample sequence.

In another aspect, the invention includes a probe for analyzing a nucleic acid, e.g. for detecting a SNP in a oligonucleotide, for example, a piece of genomic DNA. The probe includes:

a linear or circular single stranded oligonucleotide having:

i) optionally, an universal rolling circle amplification primer sequence;

ii) optionally, a polymorphism specific to the sequence;

iii) a region which is complimentary to a portion of the oligonucleotide, which preferably is twelve to twenty nucleotides, directly adjacent to, but not including the SNP; and

iv) a second region which is complimentary to a second portion of the oligonucleotide which contains the SNP and one or more, but preferably four or five complimentary nucleotides.

In a preferred embodiment, the linear or circular single stranded oligonucleotide contains a structural element that cleaves the rolling circle amplification product, for example, a self complimentary hair pin.

In another aspect, the invention includes a kit or reaction mixture having a probe described herein as an additional short nucleotide is which is complimentary to several nucleotides directly adjacent to the SNP, and has a nucleotide directly adjacent to the SNP but not complimentary to it.

The nucleic acids, e.g., probes and primers, arrays, and other reagents or devices disclosed herein ar also within the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of a construct containing a polymorphism-specific tag sequence.

FIG. 2 is a schematic diagram of a construct containing an allele-specific tag sequence.

FIG. 3 is a schematic diagram showing a capture probe attached to a solid support.

### DETAILED DESCRIPTION

Embodiments of the invention are based on the use of circular vectors (e.g., vectors described in U.S. Pat. No. 5,714,320) to analyze a sequence. The methods described herein can be used on any method for which identification of specific nucleic acid sequences are desirable, including the identification of specific nucleotides in a nucleic acid sequence. Thus, the methods can be used to identify single-nucleotide polymorphisms (SNPs) or other mutations in DNA and RNA molecules. The methods can also be used to diagnose or stage a disease state, or predisposition to a disease or condition, and can also be used generally in expression profiling or analysis.

The detection methods described herein can include circular vectors which anneal to a target nucleic acid containing a sequence of interest. The annealed target sequence is then further amplified and characterized.

The circular vectors can be closed circular vectors, or open circular vectors which when brought into contact with the analyte, have abutting ends which can be covalently linked, e.g., ligated.

Rolling circle amplification (RCA) is used to generate many copies of an nucleic acid sequence, preferably with defined ends (e.g., as described in U.S. Pat. No. 5,714,320). The single-stranded product of rolling circle amplification can be rendered double-stranded by the annealing of un-circularized, complementary probe vector. The dsDNA RCA product can be fragmented, e.g., using a type IIS restriction enzyme, such that the DNA is cleaved in the middle, or at the ends, of the region generated by the ligation reaction. The dsDNA fragments generated by the restriction digest can be analyzed, e.g., on an array, e.g., an array of indexing linkers (see, e.g., U.S. Pat. No. 5,508,169). If the probe vector is labeled with a capture or anchoring moiety, e.g., a biotin group, then it is possible to render the dsDNA fragments generated from fragmentation of the RCA product single-stranded by thermal denaturation following the addition of capture or anchoring moiety reactive, e.g., strepavidin-labeled, substrates, e.g., magnetic beads or a solid support. The single-stranded DNA fragments can be analyzed on a Cantor-type array, as described in e.g., U.S. Pat. No. 5,503,980.

In other embodiments, the captured DNA fragments are analyzed using mass spectrometry. The target DNA is applied to a multiplicity of wells and a population of RCA vectors is added to each well. The RCA products are analyzed using mass spectrometry following fragmentation, where the amplification of specific RCA vectors is determined by differences in molecular weight of the RCA product fragments. Multiple RCA vectors can be analyzed simultaneously in a single reaction using this approach.

### Positional Arrays

Positional arrays suitable for the present invention include high and low density arrays on a two dimensional or three dimensional surface. Positional arrays include nucleic acid molecules, peptide nucleic acids or high affinity binding molecules of known sequence attached to predefined locations on a surface. Arrays of this nature are described in numerous patents which are incorporated herein by reference. These include, e.g., Cantor, U.S. Pat. No. 5,503,980; Southern, EP 0373 203 B1; Southern, U.S. Pat. No. 5,700,637 and Deugau, U.S. Pat. No. 5,508,169. The density of the array can range from a low density format, e.g., a microliter plate, e.g., a 96- or 384- well microliter plate, to a high density format, e.g. 1000 molecules/cm², as described in, e.g., Fodor, U.S. Pat. No. 5,445,934.

The surface on which the arrays are formed can be two dimensional, e.g., glass, plastic, polystyrene, or three dimensional, e.g. polymer gel pads, e.g. polyacrylamide gel pads of a selected depth, width and height.

In preferred embodiments, the target or probes bind to (and can be eluted from) the array at a single temperature. This can be effected by manipulating the length or concentration of the array or nucleic acid which hybridizes to it, by manipulating ionic strength or by providing modified bases.

### Proximity Methods

In some embodiments, nucleic acid products are detected using proximity-based methods. Proximity methods include those methods whereby a signal is generated when a first member and second member of a proximity detection pair are brought into close proximity.

A “proximity detection pair” will have two members, the first member, e.g., an energy absorbing donor or a photosensitive molecule and the second member, e.g., an energy absorbing acceptor or a chemiluminescer particle. When the first and second members of the proximity detection pair are brought into close proximity, a signal is generated.

### Fluorescence Resonance Energy Transfer (FRET)

Fluorescence resonance energy transfer (FRET) is based on a donor fluorophore that absorbs a photon of energy and enters an excited state. The donor fluorophore transfers its energy to an acceptor fluorophore when the two fluorophores are in close proximity by a process of non-radiative energy transfer. The acceptor fluorophore enters an excited state and eliminates the energy via radiative or non-radiative processes. Transfer of energy from the donor fluorophore to acceptor fluorophore only occurs if the two fluorophores are in close proximity.

### Homogeneous Time Resolved Fluorescence (HTRF)

Homogeneous time resolved fluorescence (HTRF) uses FRET between two fluorophores and measures the fluorescent signals from a homogenous assay in which all components of the assay are present during measurement. The fluorescent signal from HTRF is measured after a time delay, thereby eliminating interfering signals. One example of the donor and acceptor fluorophores in HTRF include europium cryptate [(Eu) K] and XL665, respectively.

### Luminescent Oxygen Channeling Assay (LOCI)

In the luminescent oxygen channeling assay (LOCI), the proximity detection pairs includes a first member which is a sensitizer particle that contains phthalocyanine. The phthalocyanine absorbs energy at 680 nm and produces singlet oxygen. The second member is a chemiluminescer particle that contains olefin which reacts with the singlet oxygen to produce chemiluminescence which decays in one second and is measured at 570 nm. The reaction with the singlet oxygen and the subsequent emission depends on the proximity of the first and second members of the proximity detection pair.

### Gel Pad Arrays

Gel pads, including arrays of gel pads, can be prepared by a variety of methods, some of which are known in the art. Examples of these methods are provided in, e.g., Timofeev et al., Nucleic Acids Research (1996), Vol. 24, 3142-3148, Drobyshev et al., Gene (1997) 188: 45-52; Livshits et al., Biophysical Journal (1996) 71:2795-2801; Yershov et al., Proc. Natl. Acad. Sci. USA (1996) 93:4913-4918; Dubiley et al., Nucleic Acids Research (1997), Vol. 25, 2259-2265; and U.S. Pat. No. 5,552,270 by Khrapko et al. Each of the foregoing is incorporated herein by reference. Gel pad arrays are the preferred positional arrays for use in the methods described herein.

In some embodiments, a sample which contains a target analyte, e.g., a polynucleotide, such as a sample which contains genomic DNA, is loaded into a gel pad. An array of gel pads on a first solid support can be employed to perform an analysis on a plurality of samples, or a plurality of probes to detect a plurality of characteristics, e.g., SNPs, of a sample or samples. The genomic DNA is preferably digested, e.g., with a restriction enzyme, to provide shorter fragments of DNA which can easily diffuse into the gel pad(s). The gel pad composition and/or the size of fragments can be selected to permit the target polynucleotides to diffuse into the gel pad, and/or to prevent larger pieces of, e.g., genomic DNA from diffusing into the gel pad. The volume of the gel pad(s) is preferably less than about 1 microliter, more preferably less than about 500, 100, 50, 10, 5, 1, 0.5, or 0.1 nanoliters per gel pad. Volumes in this range permit the diffusion of reactants and target to occur in a conveniently short time period (e.g., preferably less than 5, 2, 1, 0.5, or 0.1 minutes). After the sample polynucleotide has diffused into the gel pad, the remaining sample can be washed away.

An “array” can be any pattern of spaced-apart gel pads disposed on a substrate. Arrays can be conveniently provided in a grid pattern, but other patterns can also be used. In preferred embodiments, a gel pad array includes at least about 10 gel pads, more preferably at least about 50, 100, 500, 1000, 5000, or 10000 gel pads. In some embodiments, the array is an array of gel pads of substantially equal size, thickness, density, and the like, e.g., to ensure that each gel pad behaves consistently when contacted with a test mixture. In certain embodiments, however, the pads of a gel pad array can differ from one another; e.g., a mixed gel pad array can be constructed which includes more than one size or type of gel pad, e.g., gel pads made of different gel materials, or which entrap different species such as reagents or polynucleotide probes. In certain preferred embodiments, gel pads in an array are less than about 1 mm in diameter (or along a side, e.g., in the case of square gel pads), more preferably less than about 500 microns, still more preferably less than about 100, 75, 50, 25, 10, 5, or 1 micron in diameter.

A gel pad can have any convenient dimension for use in a particular assay. In preferred embodiments, a gel pad is thin enough, and porous enough, to permit rapid diffusion of at least certain reaction components into the gel pad when a solution or suspension is place din contact with the gel pad. For example, in one embodiment, a gel pad array for use in sequencing by hybridization permits polynucleotide fragments from a sample mixture to diffuse (within a conveniently short time period) into the gel pads and hybridize to oligonucleotide capture sequences disposed within the gel pads. In certain preferred embodiments, a gel pad (e.g., in an array of gel pads) has a thickness of at least about 1, 5, 10, 20, 30, 40, 50 or 100 microns. In certain preferred embodiments, a gel pad (e.g., in an array of gel pads) has a thickness of less than about 1 millimeter, 500 microns, 200, 100, 50, 40, 30, 20, 10, 5, or 1 microns.

In preferred embodiments, a first gel pad (or each the first array of gel pads) includes a first primer, e.g., a first PCR primer. The first primer is preferably complementary to at least a portion of the sample polynucleotide (or to its complement). The first primer is preferably immobilized in the first gel pad to prevent migration of the primer out of the gel pad. The immobilization can be permanent or reversible, and can be covalent or non-covalent.

A second gel pad, or second array of gel pads, can be provided on a second support. The second gel pad includes a second primer, e.g., a PCR primer, which can he complementary to a polynucleotide complementary to the sample polynucleotide. Thus, the first and second primers are preferably selected to form a pair or set of PCR primers suitable to provide amplified polynucleotides which correspond to either or both of the sample polynucleotide and its complementary strand. At least a fraction of the second primer molecules are immobilized in the second gel pad; the immobilization can be permanent or reversible; covalent or non-covalent.

In a preferred embodiment, a fraction of the first and/or second primer molecules are not immobilized, so that this fraction of the primer molecules is available to diffuse into the first gel pad when the pads are brought into contact with each other.

Either the first or the second gel pad contains reagents suitable for performing a polymerase reaction, e.g., a polymerase (preferably a thermostable polymerase suitable for thermal cycling, such as Taq polymerase), nucleotide bases (dNTPs), appropriate buffers and salts, and the like. The reagents can be added to the pads before the target is added, or after the target is added. The pads can be prepared and stored with the reagents already added, thereby providing a convenient kit for performing assays and the like. Any necessary reactants can be provided by contacting one or both of the first and second gel pads with a reaction mixture which includes the reagents, and permitting the reagents to diffuse into the gel pads. Conditions for performing polymerase reactions are well known for solution-phase reactions and can be readily adapted for gel-phase reactions according to criteria which will be apparent to the skilled artisan in light of the teachings herein.

The first and second gel pads are brought into communication, e.g., into physical contact, with each other to permit reaction components, such as non-immobilized primers, to diffuse from one gel pad to the other. The pads (or arrays of gel pads) can be brought into contact by placing the solid supports on which they are disposed into close juxtaposition. Before or after contact, the pads can be washed with wash solutions or buffers, or reaction mixtures, to remove undesired components or add reagents for reaction.

In certain preferred embodiments, an electric current is passed through the opposed gels pads. For example, the substrates can be provided with electrical contact points which function to connect an electrical potential to each of the pads. Thus, for example, the first substrate can be provided with an electrical contact for each gel pad (e.g., of a gel pad array) disposed on the first substrate, and the second substrate can be provided with electrical leads in electrical contact with each gel pad (e.g., of a gel pad array) provided on the second substrate. The electrical contacts and leads are connected to a source of electrical potential configured such that when the gel pads on opposing surfaces are brought into contact with each other, an electrical current can be passed through each of the gel pads (i.e., a circuit is completed).

In another embodiment, an electrical potential can be used to promote a chemical reaction in a gel pad. For example, electrochemical reductive or oxidative cleavage reactions are well known in the art, and can be promoted by application of an appropriate electrical potential to a reaction mixture. Thus, application of a potential to a gel pad can be used to promote an oxidative or reductive reaction 4n the pad. Any gel pad in an array of gel pads can be selectively targeted for reaction by applying a potential to that gel pad (and its opposed gel pad on the opposing substrate), preferably without subjecting other gel pads in the array to the electrical potential.

In still another embodiment, an electrical potential can be used to promote the migration of a reaction component into a gel pad. Thus, selected gel pads of an array of contacted, opposed gel pads, can be subjected to an electrical potential to promote the migration of reaction components into, or out of, the gel pad (and, preferably, into the opposed gel pad and/or a reaction mixture which surrounds the gel pad).

In still another embodiment, an electrical potential can be used to promote a change in the characteristics of the gel pad. For example, so-called “intelligent gels” have been described. These intelligent gels are responsive to electrical currents, e.g., the gel shrinks or swells in response to electrical potential. Thus, application of an electrical potential can be used to cause a gel pad to shrink, which could interrupt the electrical current. Thus, a form of feedback control can be attained, e.g., to prevent gel pads from contacting an opposing gel pad, or to maintain opposed gel pads in contact with each other for any desired period of time.

A PCR amplification can then be performed by subjecting the gel pads to thermal cycling as is known in the art. The thermal cycling can be performed with the gel pads in direct contact. Alternatively, the gel pads can be separated once the appropriate reaction components have diffused into each gel pad, and each separated gel pad (or array) can be subjected to thermal cycling. In certain embodiments, it is preferred to separate the pads, to prevent thermal stresses from causing cracking or other loss of integrity of a pad. If desired the gel pads can be brought back into contact after any round of thermal cycling.

During thermal cycling it is preferable to seal the gel pads to prevent evaporation of liquid. Sealing can be provided by placing the gel pads in a hermetically sealed container such as a chamber, or alternatively by covering the gel pad with a non-evaporating liquid such as an oil. The oil can be removed after cycling, e.g., by washing with a suitable solvent or detergent solution. Between cycling rounds, the pads can be exposed to fresh reagent solutions, if necessary, e.g., by opening the sealed chamber or by washing away a protective oil layer.

After sufficient rounds of thermal cycling have occurred, the gel pads can be washed to remove excess reagents. The washing step is performed under conditions which do not remove the immobilized (and now extended) primers, but which do remove non-immobilized primers, and other reactants.

The gel pads can then be analyzed to determine a characteristic, e.g., an SNP of the immobilized primers. Either gel pad can be analyzed, or both can be analyzed to provide a redundant analysis (e.g., the analysis of one strand can be compared to the analysis of the other strand to ensure accurate results). A gel pad containing a strand (either target or complement) can also be retained as a backup or for record keeping purposes. In one embodiment, the analysis includes: providing primers which bind adjacent to an SNP, dideoxynucleotides (ddNTPs), and a polymerase (which can be the same polymerase used for the PCR reaction). The ddNTPS are preferably labeled, e.g., with distinct, distinguishable fluorescent labels. The primers are then extended with the polymerase, and the gel pads are washed to remove the unincorporated reactants. The base present at the SNP can then be determined by detection of the labeled ddNTP present in the gel pad.

It will be appreciated from the foregoing that arrays of gel pads can be used, with a first array of gel pads being provided on a first substrate (e.g., a glass plate) and second array of gel pads being provided on a second substrate. The first and second arrays are preferably prepared in registration, e.g., having the same size, number, and separation of gel pads, so that when the two substrates are brought into close contact, each gel pad of the first array is in contact with a gel pad of the second array.

It will also be appreciated that more than two arrays of gel pads can be brought into contact. For example, first and second gel pads (or arrays of gel pads) can be provided on a porous substrate which has a hole or plurality of holes therethrough. The first and second gel pads can be positioned on the respective substrates adjacent to a hole. A third array of gel pads can then be provided on an (array of) members which fit in engaging relation with the hole(s) of the first and/or second substrate, such that a gel pad disposed on a member can engage the first and second gel pads in communication to permit a reaction to occur in any or all of the gel pads.

In preferred embodiments, the gel pads contain a primer. The primer-containing gel pads is then contacted with a gel pad (or array) which includes reactants for a “proofreading” detection system, i.e., a system which includes enzymes which can ensure the fidelity of the detection format (the reactants can optionally be added separately). In certain embodiments, at least one probe of the proofreading probes is provided with a “handle” which can be bound by a specific-binding “hook”, and the proofreading gel pad includes a “hook” for immobilizing a primer, such as strepavidin (e.g., for binding to biotin). For example, a DNA ligase can be used to ensure that hybridized probes have perfect complementary to a portion of a sample or primer DNA. After the proofreading reaction(s) is complete, the proofreading probe(s), which include a protected (masked) biotin label, are deprotected (e.g., by exposure to light to deprotect a photodeprotectable biotin moiety). The probe is then captured by streptavidin in the proofreading gel pad (or array), which is then washed to remove extraneous reactants, and the immobilized probe is detected (e.g., with a color charge-coupled device (CCD) camera) to detect differentially colored fluorescent labels on the probe(s)

### Rolling Circle and Additional Amplification

Rolling circle amplification (RCA), in combination with detection technologies known in the art, can be used to amplify nucleic acids which have annealed to a target sequence. In some embodiments, additional rounds of RCA amplification, or RCA amplification in conjunction with other amplification procedures such as PCR or NASBA may be desirable for achieving specific detection, e.g., in some cases of an allele in genomic DNA. Thus, regions of genomic DNA containing sites of polymorphisms can be amplified by PCR prior to contact with circular templates. After PCR the unincorporated primers and dNTPs can be destroyed enzymatically using, e.g., exonuclease and shrimp alkaline phosphatase, which can then be destroyed by heating at 80° C.

### Microplate Protocol

In the case of detection of polymorphisms in candidate genes, sample, e.g., PCR products, can be distributed to multiple wells, the number depending on the number of polymorphisms in the amplified region to be analyzed—two wells can be for each polymorphism (e.g., 192 biallelic polymorphisms on a 384-well plate). In the case of detection of polymorphisms in a biallelic SNP map, each PCR reaction can be divided between two wells.

An open circle probe can be added to each well, with a separate probe provided for each allele of each polymorphism. If both strands are to be analyzed, twice as many probes and twice as many wells are be required. The probes which anneal are ligated, and RCA is per-Formed with labeled dNTPs, preferably two labels, so that both labels are incorporated into the RCA product. The labels can, e.g., prompt fluorescence FRET pairs or haptens to which HTRF or LOCI labels could be bound after the RCA. Alleles are determined by comparing the signals in the two wells containing the two corresponding circular probes.

No separation is required in this assay. In addition, handling of the liquid can be handled with devices known in the art, e.g., a MultiProbe with a thermocycler.

### RCA-based Amplification and Detection

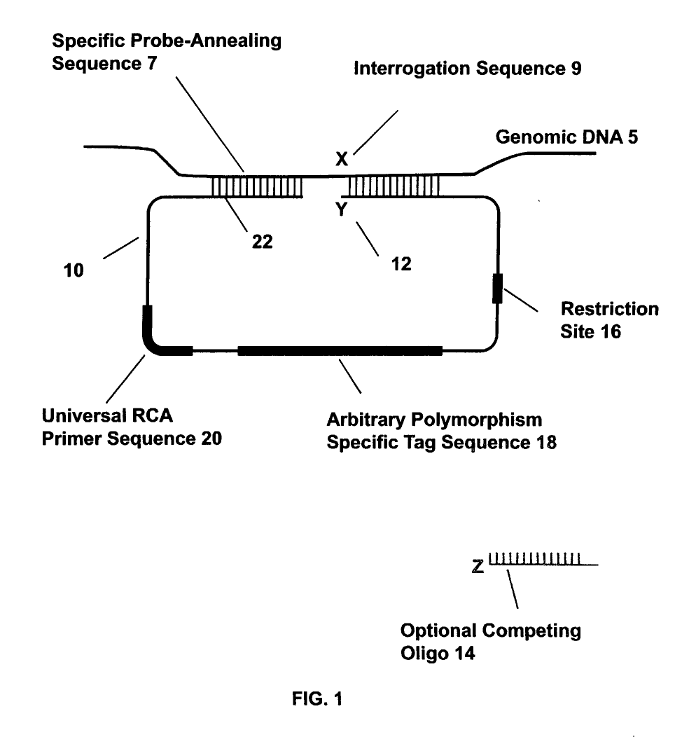

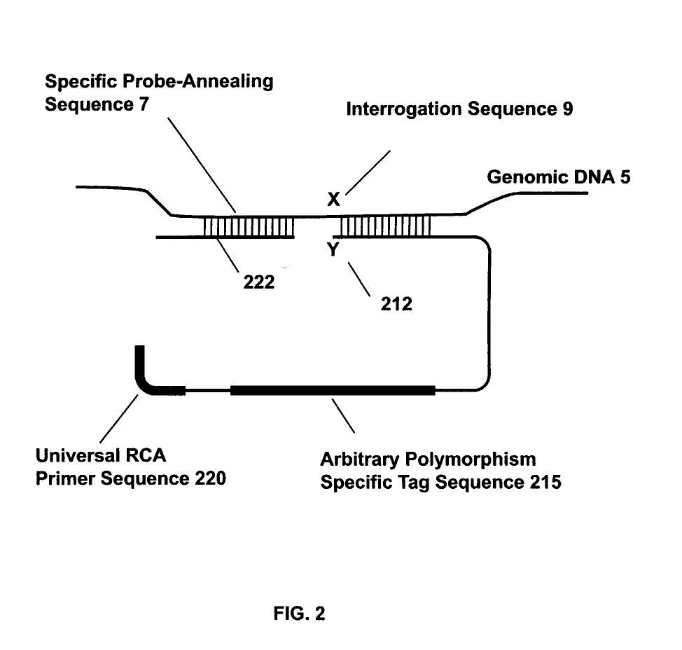

Examples of probes suitable for use with methods of the invention are provided in FIGS. 1 and 2. FIG. 1 shows a linear nucleic acid probe 10, also known as a “padlock probe”. The probe 10 is shown with an interrogation region 12 at its 5′ end. The interrogation region 12 contains about 5 bases of sequence complementary to a sequence in a target sequence 5. The target sequence 5 contains a specific probe-annealing sequence 7 and interrogation sequence 9. The target sequence 5 can be any polynucleotide, e.g, DNA, RNA, cDNA, synthetic or isolated from an organism, or virus. In some embodiments the nucleic acid is amplified prior to incubation with the linear nucleic acid probe, e.g., the target sequence 5 can be PCR-amplified genomic DNA.

The interrogation sequence 9 in the target sequence can include a region known to contain, or suspected of containing, a polymorphic region such as a single nucleotide polymorphism (SNP). In FIG. 1, the polymorphism in the target nucleic acid sequence is denoted by an “X”.

If complementary sequences are present between the interrogation region 12 and the interrogation sequence 9 target sequence 5, the interrogation region 12 hybridizes to the target and be stabilized by contiguous base stacking. The end of the probe 10 corresponding to the interrogation region 12 can be ligated to the other end of the probe 10 if its terminal nucleotide (“Y”) forms a complementary base pair with the site of the polymorphism (“X”) in the interrogation sequence 9 of the target sequence 5.

In contrast, the interrogation region 12 is much less likely to stably hybridize to the sequence 5 if there is a mismatch between the terminal nucleotide “Y” and the nucleotide at position “X”. In the latter case, the mismatch between the terminal nucleotide in the interrogation region and the target nucleic acid sequence will preclude ligation of the ends of the probe molecule 10.

An optional competing oligonucleotide 14 having a terminal nucleotide (“Z”) can be included in the reaction. The competing oligonucleotide 14 is complementary to a second allele of a biallelic polymorphism “X” and is preferably about 5 nucleotides in length. The competing oligonucleotide 14 inhibits hybridization of the interrogation region of the probe 10 if the correct base pairing is between “X” and “Z”, rather that “X” and “Y”. Thus, if “Y” is the complementary base to “X”, then the probe 10 is ligated and circularized to form a circle. If “Z” is the complementary base to “X”, the competing oligonucleotide 14 anneals to the target nucleotide sequence. No circular product results from this ligation product, and the product is not a substrate for rolling circle amplification.

The presence of the competing oligonucleotide 14 is not necessary if the ligation reaction is sufficiently sensitive to a mismatch at the site of the polymorphism. However, inclusion of the competing oligonucleotide 14 may nevertheless be desirable because it can significantly enhance the fidelity of the reaction.

The probe 10 optionally includes a restriction endonuclease recognition site 16. In some embodiments the restriction endonuclease will cleave the single-stranded nucleic acid template. In other embodiments, the restriction endonuclease will cleave upon annealing of a complementary nucleotide sequence, e.g., a complementary oligonucleotide such as a short universal oligonucleotide.

The complementary oligonucleotide can be added to form a double-stranded region at the restriction site 16. In some embodiments, the restriction site 16 is a site recognized by a type IIS restriction endonuclease. In some embodiments, the restriction site 16 may form a self-complementary hairpin so that individual copies of the RCA product can be cleaved by simply adding the appropriate restriction enzyme.

The probe also includes an arbitrary polymorphism-specific tag sequence 18 which can be used to specifically identify the probe 10. The unique tag sequence 18 is specific for each polymorphism in a pool. The length, base composition and sequence of the tag sequence 18 are chosen to permit highly specific, unambiguous hybridization of a large number of probes to complementary capture probes on a generic oligonucleotide array, as is described below. The tag sequences are preferably designed and selected for unambiguous discrimination and capture on an array.

In preferred embodiments, the specific tag sequence 18 is located close to the restriction endonuclease recognition site 16. Cleavage of the RCA product with a Type IIS restriction endonuclease results in the tag being positioned on the end of the RCA product, and hence allows for capture of the cleaved RCA product on a Cantor-style array, as is described below.

The probe 10 may also contain a RCA primer sequence 20, which allows for priming of rolling circle amplification of the circularized probe 10 upon annealing of a complementary RCA primer. The RCA product formed by the rolling circle amplification is labeled by including one or more labeled dNTPs in the amplification reaction.

The probe 10 has a terminal sequence 22 of approximately 15 nucleotides at its 3′ end. The terminal sequence 22 provides highly specific annealing of the probe to the specific probe-annealing sequence 7 of the target nucleic acid 5 at a location adjacent to the polymorphic site in the target nucleotide sequence 5. The length of the terminal sequence 22 can be adjusted so that all probes in a collection of probes have approximately the same melting temperature.

A probe suitable for use in nucleic acid sequence sequencing is shown in FIG. 2. A target nucleic acid 5, indicated as a PCR-amplified genomic DNA, has a specific probe annealing sequence 7 and an interrogation sequence 9.

The probe 200 includes an interrogation region 212, which includes an interrogation nucleotide “Y”. The interrogation nucleotide “Y” is either A,C, G, or T. The probe 200 also includes an RCA primer recognition sequence 220, and a terminal sequence region 222. Each of these elements are analogous to the corresponding regions in FIG. 1.

The probe in FIG. 2 also contains an allele-specific tag sequence 215, which has a sequence that is specific for the corresponding interrogation nucleotide. Thus, the allele-specific tag sequence 215 allows for the determination of a particular nucleotide “X” in a target nucleic acid sequence upon hybridization of the interrogation region 212 to the target nucleic acid sequence 5.

While the probes depicted in FIGS. 1 and 2 are shown with the interrogatory regions and terminal sequences at the 5′ and 3′ ends of the molecules, respectively, the probes may alternatively be designed in the reverse orientation, i.e., with the interrogatory region at the 3′ end and the terminal sequence at the 5′ end.

Rolling circle-based amplification using a circularized probe molecule in the presence of polymerase and dNTPs, at least one, and more preferably, two, three or even all four of which are labeled with a label, e.g., a fluoroophor, hapten, or radioactive label. RCA-mediated amplification results in about a 1000-fold amplification of the circularized probe.

A type II S restriction endonuclease in the presence of a complementary oligonucleotide can cleave the RCA products, leaving the nucleotide corresponding to the polymorphic site at the 5′ end of the single-stranded RCA products. The cleaved products will preferably be about 40-45 nucleotides long.

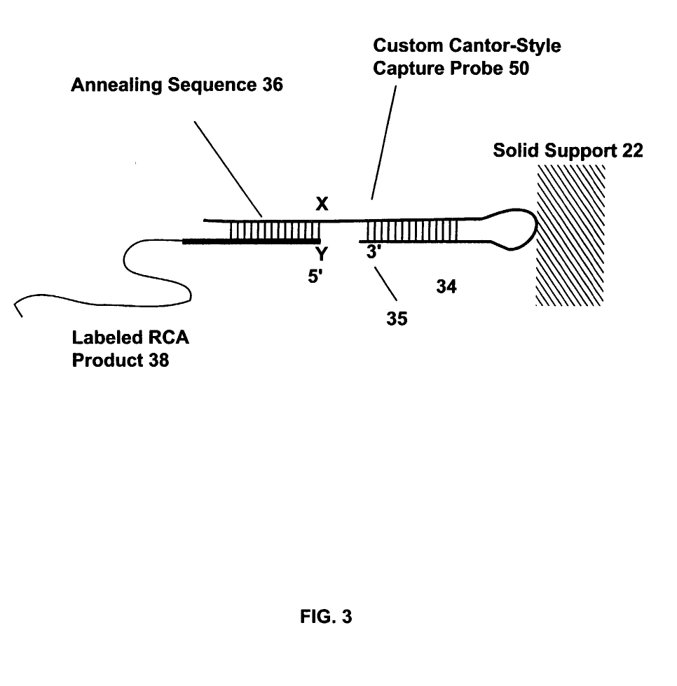

The RCA products can be perfused over an array of custom Cantor-style probes having 5′ overhangs, e.g., as described in U.S. Pat. No. 5,503,980 and as shown schematically in FIG. 3. FIG. 3 demonstrates a Cantor-style, partially duplex capture probe 30 attached to a solid support 32. The capture probe 30 includes a double-stranded region 34 and a single-stranded region annealing sequence 36 at its 5′ end. The single stranded region 36 includes a nucleotide “Y” at the 3′ end of the single-stranded region.

The RCA product 38, which has been cleaved immediately 5′ to the site of the polymorphism, includes an interrogatory nucleotide “X” at its 5′ end. The RCA product will ligate to the capture probe 30 at location 25 only if it was amplified from a padlock probe, e.g., those described in FIGS. 1 and 2, that was exactly complementary to the genomic target at the site of the polymorphism. Thus, the RCA product 38 will anneal to the Cantor-style probe 30 only if nucleotides “X” and “Y” pair.

For each allele of each polymorphism there is a corresponding immobilized probe in a gel pad or array cell that is complementary to the 5′ end of the corresponding RCA product, i.e., for 1,000 biallelic polymorphisms there will be at least 2,000 array elements. All probes for a given polymorphism will be identical except for the base at the site of the polymorphism, i.e., nucleotide “Y” in FIG. 3.

While the probe shown is shown in FIG. 3 as a single oligonucleotide with a hairpin structure that is immobilized on the solid support, the arrays can alternatively be made with single stranded at their 3′ ends. The shorter oligonucleotide, which can be a universal oligonucleotide, can be annealed at the time of the analysis.

After hybridization and washing, the RCA products are ligated to the capture probes, and any products not ligated can be washed away at a high temperature. Target nucleic acids containing specific sequences, e.g., alleles carrying specific polymorphisms, are determined by noting which of the microarray locations specific for a given polymorphism contain RCA products.

The invention also includes a set of two or more such probes, preferably as elements of a positional array, e.g., a three dimensional or gel pad array. A large number of probes can be annealed specifically to their targets in the same tube or well under the same conditions.

### Microarray Protocol

For detecting polymorphisms in candidate genes, polymorphic regions of any size can optionally be amplified using PCR prior to performing RCA-based analysis. PCR amplification can occur in a single tube or well, and more than one polymorphic region can be amplified by multiplex PCR in the same tube. In the case of detection of polymorphisms in a biallelic map, many PCR reactions can be pooled, thereby minimizing the number of PCR reactions performed.

Performing PCR analysis in conjunction with RCA analysis can lessen the amount of PCR amplification required. Thus, there is less chance of PCR reagents being exhausted during the reaction.

The pooled PCR products cam be divided into two portions, when biallelic polymorphisms are examined. If more than two alleles for the polymorphisms are present, or if the presence of bases that are not expected to be alleles are examined, as negative controls, the products are divided into four portions. For each polymorphism to be interrogated, one allele-specific probe is added to each of the aliquoted portions. Large pools of padlock probes can be added to the PCR products. The number of probes can be, e.g. 10, 100, 500, 1,000, or 5,000.

In some embodiments, the probes have 17 to 25 bases of sequence complementary with their targets, and the lengths of the regions of complementarily are designed so that all the probes have about the same melting temperature.

In the presence of a universal primer, polymerase and a labeled, e.g., fluorescent, dNTP, intensely labeled. e.g., fluorescent, a fluorescent nucleotide having a characteristic color can be used for each of the two (four) reactions or alleles. For 60,000 biallelic SNP markers, 48 PCR pools can be created, each with 1250 PCR products. The PCR products can optionally be multiplexed, in whole or in part, and the RCA reaction can be performed on a 96-well plate.

After completion of the RCA reaction, the wells corresponding to the alleles for each pool of probes are combined and hybridized on a generic array. One array element is required for each polymorphism. The allele, or alleles, if heterozygous of each polymorphism can be determined from the color of the array element. Thus, for example, all of the 60,000 SNP markers in the biallelic map require 48 generic 1250-element arrays. Interrogation of both DNA strands requires twice the number of arrays.

When gel pads are used, the RCA products are cleaved into short fragments with a restriction enzyme. An important advantage of the RCA method is that, because of the amplification, a high concentration of a small molecular weight target molecule hybridizes to the array. Thus, the reaction can proceed quickly, and the resulting signal is quite strong, especially since the RCA products are intensely labeled.

RCA Probes for Polymorphism Detection

SNP analysis of a large number of polymorphisms in a biallelic SNP map will sometimes require a number of amplification reactions. Amplification, e.g., PCR (or NASBA) can be performed in gel pads with probes as is described above. In this case the probe can be annealed to the immobilized amplification product in the gel pad.

Analysis of multiple polymorphisms in a sample of genomic DNA can be performed in a two-step process. First, a pool of probes is incubated with the target sample in a single tube. The annealing, ligation, and rolling circle amplification (RCA) steps to be described below are performed in this tube. Second, the RCA products are perfused over a partially duplex oligonucleotide array on which allele-specific RCA products will be captured by hybridization and ligation. Alleles corresponding to various polymorphisms are determined by noting the microarray locations in which RCA products are present.

A variety of targets can be used, e.g., single-stranded PCR products, denatured double-stranded PCR, or unamplified genomic DNA. For each of the polymorphisms to be analyzed, which can number around 1,000, there are 2-4 probes which differ only by the base at their 5′ termini. Probes will anneal to their target sequences as is indicated in FIG. 3.

Probes for alleles corresponding to some or all polymorphism sites on the array are applied to the array. The probes contain allele-specific tags, of which there ware a total of only four—one for each base A, C, G, T. Competing pentamers are not used, since both (all four) alleles are present during the hybridization and ligation. As can be seen in FIG. 2, a restriction site is not necessary in a probe for determining a DNA sequence. In fact, cleavage of the RCA product would be undesirable, since small fragments could diffuse from the gel pads.

In this embodiment, there are only non-fluorescent dNTPs present during the RCA reaction. The RCA products are labeled with generic allele-specific hybridization probes labeled with different color fluorophors, of which there are only four (A, C, G, T). The sequences of the allele-specific tags and the probes can be designed to provide very unambiguous differentiation of the four possible alleles (assuming the four fluorescent dyes could be adequately separated). There is great flexibility in the labeling of the probes (compared to the use of fluorescent ddNTP terminators).

### Identification of RNA (RNA Profiling) and Seguencing of Mutations and SNPs Using Rolling Circle Amplification and Capture Arrays

A pre-formed circular vector is applied to single-stranded cDNA in order to identify and quantitate the RNA molecules in a population of RNA molecules obtained from normal and disease cells. A population of circular vectors is applied to gel pad arrays containing cDNA or RNA, columns and affinity chromatography using cDNA or RNA (see, e.g., U.S. Pat. No. 5,714,320) or arrays of cDNA or RNA attached to a solid support (see, e.g., U.S. Pat. No. 5,503,980).The circular vectors include:

1) A region of random DNA sequence (e.g., 5-50 bases, preferably 12 bases);

(2) A region containing a recognition sequence for a type IIS restriction enzyme that cleaves in the middle of the region of random DNA sequence (note: this region may be designed to form a hairpin or other structure as described in, e.g., U.S. Pat. No. 5,714,320);

(3) Additional DNA sequence that is, ideally, no: complementary to any of the target nucleic acid sequences (RNA or cDNA) such that the complete vector contains between 50-1500 bases.

Those circular vectors that recognize sequences in the target are separated from the population of circular vectors added to the target nucleic acids. Background hybridization can be minimized by including linear DNA that contains all of the vector sequence except for the region of random DNA. The isolated circular vectors are amplified using rolling circle amplification (e.g., in the presence of a fluorescent nucleotides), the DNA is cleaving, e.g., using a restriction enzyme, and the resulting fragments are analyzed, e.g., interrogated on an indexing linker array (if dsDNA) see, e.g., U.S. Pat. No. 5,508,169, or a Cantor-type array (if ssDNA) see, e.g., U.S. Pat. No. 5,503,980. Preferably the analysis is performed in a 3 dimensional gel pad array, see, e.g., U. S. Pat. No. 5,552,270.

In another embodiment, circular vectors (as above) are used to identify the presence of mutations and SNPs by having a region of the circular DNA complementary to a mutation or SNP such that the circular DNA specifically binds to the mutation or SNP. Circular vectors complementary to a mutation or SNP will be isolated through application to a population of target DNA molecules (cDNA or RNA) e.g., bound to a solid support, a gel pad or a bead. The target DNA can be present as either an ordered array of distinct molecules, or as a non-ordered array of molecules on a solid support, a gel pad or a bead. The resulting vectors are amplified by rolling circle amplification (e.g., in the presence of a fluorescent nucleotides), and can be fragmented by restriction enzymes, and analyzed, e.g., on an indexing linker (if dsDNA) see, e.g., U.S. Pat. No. 5,508,169 or a Cantor-type array (if ssDNA) see, e.g., U.S. Pat. No, 5,503,980.

Vectors can be separated into pools to prevent hybridization between the vectors (dsDNA probes should be avoided) and to maximize hybridization fidelity in any method described herein. The vector pools are applied to anchored target nucleic acid (genomic DNA, amplified DNA, cDNA or RNA) and those that hybridize to sequences in the target nucleic acid are isolated from the pool (conditions selected that maximize hybridization fidelity for each vector pool). The identity of the isolated vectors is determined by RCA, where the isolated oligonucleotide probes act as both a “positioning oligo” and an RCA primer (e.g., as in U.S. Pat. No. 5,714,320). The DNA derived from rolling circle amplification (in the presence of a fluorescent nucleotides) is cleaved using a restriction enzyme, and the resulting fragments can be interrogated on an indexing linker array (if dsDNA) see, e.g., U.S. Pat. No. 5,508,169 or a Cantor array (if ssDNA) see. e.g., U.S. Pat. No. 5,503,980.

### DNA Sequencing

A linear DNA vector probe is designed with two, random, e.g., 5 mer, sequences in either end of the vector. There are 1024 possible 5mer sequences, so this entails the synthesis of 1,048,576 linear vectors. The vectors will share one or a small number of common backbones, where each backbone can include a type IIS restriction site and a priming site for DNA synthesis. The vectors should be grouped such that the random 5 mers in a given group of vectors can not be brought together by the common backbone sequence. The sequence of the target nucleic acid will then facilitate the circularization of a subset of the probe vectors, with each circularized probe vector representing a short contiguous, e.g., 10 base pair, stretch of target DNA. The DNA is amplified using RCA in the presence of fluorescent nucleotides. The single-stranded product of rolling circle amplification is rendered double-stranded by the annealing of un-circularized, complementary probe vector. The dsDNA RCA product is analyzed. It can be fragmented, e.g., using a type IIS restriction enzyme such that the DNA is cleaved in the middle of the short region generated by the ligation reaction. The dsDNA fragments generated by the restriction digest are analyzed, e.g., on an array of indexing linkers (see, e.g., U.S. Pat. No. 5,508,169). If the probe vector is labeled with a capture moiety, e.g., biotin group, then it is possible to render the dsDNA fragments generated from fragmentation of the RCA product single-stranded by thermal denaturation following the addition of capture moiety reactive, e.g., substrate, e.g., strepavidin-labeled substrate, e.g., magnetic beads or solid support. The single-stranded DNA fragments can then be analyzed on a Cantor-type array. The DNA sequence of the target DNA is reconstructed using overlap analysis according to the procedure of Drmanac et al. (see, e.g., U.S. Pat. Nos. 5,464; 5,492,806; 5,202,231; and 5,695,940).

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of analyzing a polynucleotide sequence in a sample, comprising:
providing a sample polynucleotide sequence to be analyzed;
annealing an effective amount of sample polynucleotide sequence to a single-stranded circular template to yield an annealed circular template, wherein the single-stranded circular template comprises at least one copy of a nucleotide sequence complementary to the sample sequence and at least one nucleotide effective to produce a cleavage site;
providing the annealed circular template with effective amounts of a primer, at least two types of nucleotide triphosphates, and a polymerase enzyme to yield a single-stranded oligonucleotide multimer having a sequence comprising the sequence of the sample polynucleotide sequence and cleavage site;
providing an array of a plurality of capture probes, wherein each of the capture probes is positionally distinguishable from other capture probes of the plurality on the array and wherein each of the capture probes contains a region of unique sequence; and
hybridizing the amplified sample sequence with the array of capture probes, thereby analyzing the sample sequence.

2. The method of claim 1, further comprising cleaving the oligonucleotide multimer at the cleavage site to produce cleaved amplified sample nucleic acid.

3. The method of claim 2, wherein said cleaved amplified sample nucleic acid is hybridized to the array of capture probes.

4. The method of claim 1, wherein the oligonucleotide multimer is further amplified prior to hybridizing with the array of capture probes.

5. The method of claim 3, wherein the further amplification is by rolling circle amplification.

6. The method of claim 2, wherein the cleaved amplified sample nucleic acid is further amplified.

7. The method of claim 1, wherein the single-stranded circular template is prepared by a process comprising the steps of
(a) hybridizing each end of a linear precursor oligonucleotide to a nucleotide sequence in the sample polynucleotide sequence complementary to a portion of the sequence comprising the 3′ end of the linear precursor oligonucleotide and a nucleotide sequence in the sample polynucleotide sequence complementary to a portion of the sequence comprising the 5′ end of the linear precursor oligonucleotide, thereby yielding an open oligonucleotide circle wherein the 5′ end and the 3′ end of the open circle are positioned so as to abut each other; and
(b) joining the 5′ end and the 3′ end of the open oligonucleotide circle to yield a circular oligonucleotide template.

8. The method of claim 1, further including sequencing the nucleic acid.

9. The method of claim 8, wherein the sequencing step includes sequencing by hybridization or positional sequencing by hybridization.

10. The method of claim 1, wherein the method identifies a genetic event in the sample polynucleotide sequence.

11. The method of claim 1, wherein the genetic event is a single-nucleotide polymorphism.

12. The method of claim 7, wherein the method identifies a genetic event in the sample polynucleotide sequence.

13. The method of claim 7, wherein the genetic event is a single-nucleotide polymorphism.

14. The method of claim 13, wherein the genetic event is within 5 base pairs of the end of the linear precursor oligonucleotide.

15. The method of claim 12, wherein the genetic event is sufficiently close to the end of the linear precursor oligonucleotide that a mismatch inhibits DNA polymerase-based extension.

16. The method of claim 1, further comprising amplifying the sample polynucleotide sequence prior to annealing with the single-stranded circular template.

17. The method of claim 16, wherein the sample polynucleotide sequence is amplified by the polymerase chain reaction (PCR) prior to contact with the single-stranded circular template.

18. The method of claim 1, wherein the circular template includes a site for a type IIS restriction enzyme.

19. The method of claim 18, wherein the site for the type IIS restriction enzyme is positioned such that binding of a type IIS restriction enzyme at the site cleaves adjacent to the region of the single-stranded circular template which binds the sample sequence or cleaves in the region which binds the sample sequence.

20. The method of claim 1, wherein a region of the single-stranded circular template detects a genetic event.

21. The method of claim 20, wherein the region detecting the genetic event hybridizes preferentially to a sample nucleic acid having the genetic event relative to a sample nucleic acid not having the genetic event.

22. The method of claim 1, wherein each of the capture probes has a binding region for a non-specific endonuclease binding site.

23. The method of claim 22, further including:
(a) hybridizing the single stranded amplified sample sequence with the capture probe array;
(b) cleaving the single stranded amplified sample sequence/capture probe duplex with a non-specific endonuclease, to form a cleaved single stranded amplified sample sequence/capture probe duplex, such that a base corresponding to the genetic event is in the single stranded region formed by the cleavage;
(c) extending along the single strand which contains the genetic event with at least one labeled chain terminating nucleotide, such that the incorporation of a chain terminator indicates the presence of a genetic event,
thereby identifying a genetic event in the sample polynucleotide sequence.

24. The method of claim 23, further including ligating the single stranded amplified sample sequence to a strand of the capture probe.

25. The method of claim 1, wherein the sample polynucleotide sequence is DNA.

26. The method of claim 1, wherein the sample polynucleotide sequence is RNA.

27. The method of claim 1, wherein the sample polynucleotide sequence is isolated from a mammalian tissue.

28. The method of claim 27, wherein the sample polynucleotide sequence is isolated from human tissue.

29. The method of claim 1, wherein the sample polynucleotide sequence is isolated from a prenatal sample.

30. The method of claim 1, wherein the capture probes are single stranded.

31. The method of claim 1, wherein the capture probes have a structure comprising a double stranded portion and a single stranded portion.

32. The method of claim 1, wherein hybridization is detected by mass spectrophotometry.

33. The method of claim 1, wherein the amplified sample sequence has attached thereto a first member of a proximity detector pair and hybridization to the array allows the first member to be brought into proximity with a second member to provide a signal.

34. A method of analyzing a sample polynucleotide sequence comprising
(a) providing an array of a plurality of single-stranded circular templates, wherein each of the single-stranded circular templates is positionally distinguishable from other single-stranded circular templates of the array, and wherein each positionally distinguishable single-stranded circular template includes a unique region complementary to region of a sample polynucleotide sequence;
(b) contacting an effective amount of a sample polynucleotide sequence with a single-stranded circular template in said array to yield an annealed circular template, wherein the single-stranded circular template comprises at least one copy of a nucleotide sequence complementary to a region of the sample sequence;
(c) combining the primed circular template with effective amounts of a primer, at least two types of nucleotide triphosphates and an effective amount of a polymerase enzyme to yield a single-stranded oligonucleotide multimer complementary to the single-stranded circular template, wherein the oligonucleotide multimer comprises multiple copies of the sample sequence; and
(d) analyzing said sample sequence.

35. The method of claim 34, wherein said circular single-stranded template includes at least one nucleotide effective to produce a cleavage site in the oligonucleotide multimer.

36. The method of claim 35, further comprising cleaving the oligonucleotide multimer at the cleavage site to produce the cleaved amplified sample nucleic acid.

37. The method of claim 36, wherein said analyzing comprises the steps of:
(a) providing an array of a plurality of capture probes, wherein each of the capture probes is positionally distinguishable from other capture probes of the plurality on the array, and wherein each positionally distinguishable capture probe includes a unique region; and
(b) hybridizing the cleaved amplified sample nucleic acid sequence with the array of capture probes, thereby analyzing the sample sequence.

38. The method of claim 34, wherein the circular oligonucleic acid template is prepared by a process comprising the steps of:
(a) hybridizing each end of a linear precursor oligonucleotide to a nucleotide sequence complementary to a portion of the sequence comprising the 3′ end of the linear precursor oligonucleotide and a nucleotide sequence complementary to a portion of the sequence comprising the 5′ end of the linear precursor oligonucleotide, to yield an open oligonucleotide circle wherein the 5′ end and the 3′ end of the open circle are positioned so as to abut each other; and
(b) joining the 5′ end and the 3′ end of the open oligonucleotide circle to yield a circular oligonucleotide template.

39. The method of claim 38, wherein the target is amplified, e.g., by PCR, prior to contact with the circular template.

40. A method for identifying nucleotide sequences binding to a target molecule, comprising
providing a collection of circular nucleotide sequences, said collection including a sequences having a randomized sequence region and a known sequence region, wherein the known sequence region provides a binding site for an oligonucleotide primer and a cleavage recognition site;
contacting the target molecule with said nucleotide sequence;
selecting circular nucleotide sequences which preferentially bind said target molecule;
amplifying said nucleotide sequences; and
analyzing the amplified nucleotide sequences, thereby identifying circular nucleotide sequences.

41. The method of claim 40, wherein the circular molecule is prepared by a process comprising the steps of:
(a) hybridizing each end of a linear precursor oligonucleotide to a single positioning oligonucleotide having a nucleotide sequence complementary to a portion of the sequence comprising the 3′ end of the linear precursor oligonucleotide and a nucleotide sequence complementary to a portion of the sequence comprising the 5′ end of the linear precursor oligonucleotide, thereby yielding an open oligonucleotide circle wherein the 5′ end and the 3′ end of the open circle are positioned so as to abut each other; and
(b) joining the 5′ end and the 3′ end of the open oligonucleotide circle to yield a circular oligonucleotide template.

42. The method of claim 40, wherein the randomized sequence region is about 5-190 bases in length.

43. The method of claim 40, wherein the known sequence region is 5-100 bases in length.

44. The method of claim 40, wherein the known sequence region is about 8-40 bases in length.

45. The method of claim 40, wherein the target molecule is a protein.

46. The method of claim 40, wherein the target molecule is a nucleic acid.

47. The method of claim 40, wherein the selected circular nucleic acid molecules are amplified by rolling circle application.

48. The method of claim 40, wherein the circular vector is a closed circular vector.

49. The method of claim 40, wherein the circular vector is an open circular vector.

50. An array comprising a plurality of circular nucleic acid sequences, said molecules disposed at positionally distinguishable positions in the array and wherein said nucleic acid sequences comprise sequences with randomized and a nonrandomized domains.

51. The array of claim 50, wherein said circular nucleic acid molecules are about 15-1500 nucleotides in length.

52. The array of claim 50, wherein said circular nucleic acid molecules are about 24-500 nucleotides in length.

53. The array of claim 50, wherein said circular nucleic acid molecules are about 30-150 nucleotides in length.

54. The array of claim 50, wherein said circular nucleic acid molecule is DNA.

55. The composition of claim 50, wherein said circular nucleic acid molecule is RNA.

56. A method of analyzing a nucleic acid, comprising
(a) providing a first oligonucleotide;
(b) providing a second oligonucleotide, said second oligonucleotide having a first region which is complimentary to a first portion of the first oligonucleotide and a second region which is complimentary to a second portion of the first oligonucleotide;
(c) contacting the first oligonucleotide with the second oligonucleotide;
(d) linking the ends of the first oligonucleotide to form a single-stranded circular nucleic acid;
(e) providing effective amounts of a polymerase, a primer, and nucleotides to the single-stranded circular nucleic acid to form an amplified sequence comprising multimers of a sequences complementary to the single-stranded circular nucleic acid; and
(f) analyzing the resulting amplified sequence, thereby analyzing a nucleic acid.

57. The method of claim 56, wherein the first oligonucleotide contains a cleavage sequence.

58. The method of claim 56, wherein the first oligonucleotide comprises a rolling circle amplification primer sequence.

59. The method of claim 56, wherein the first oliqonucleotide is a fragment of genomic DNA.

60. The method of claim 56, wherein the first oligonucleotide is produced by polymerase chain reaction.

61. The method of claim 57, wherein the first oligonucleotide contains a sequence polymorphism.

62. The method of claim 57, wherein the first portion of the first oligonucleotide is about 12-20 nucleotides in length.

63. The method of claim 57, wherein the second oligonucleotide contains a structural element that cleaves the rolling circle amplification product.

64. The method of claim 57, further comprising the step of analyzing the products of the rolling circle amplification.

65. A probe for analyzing a nucleic acid, comprising:
a nucleic acid sequence having a first region which is complimentary to a first portion of a second nucleic acid sequence and a second region which is complimentary to a second portion of the second nucleic acid sequence, wherein the first portion and second portion of the nucleic acid sequence are positioned so that annealing of the nucleic acid sequence to the second nucleic acid sequence positions the 5′ end and the 3′ end of the second nucleic acid sequence so as to abut each other.

66. The probe of claim 65, wherein the first region is about 12-20 nucleotides from a sequence identifying a single nucleotide polymorphism (SNP).
